(19) United States
(12) Patent Application Publication
(10) Pub. No.: US 2010/0286643 A1
(43) Pub. Date: Nov. 11, 2010

(54) Sani-pantie

(76) Inventor: Alberta Thomasina Anderson, Moreno Valley, CA (US)

Correspondence Address: Alberta T. Anderson, 28107 Championship Drive, Moreno Valley, CA, 92555, US

(21) Appl. No.: 12/381,559

(22) Filed: May 11, 2009

(51) Int. Cl. A61F 13/45

(52) U.S. Cl. 604/385.01

(57) ABSTRACT

The sani-pantie provides many benefits that include convenience, safety, comfort, and less expense. Some of the benefits of the sani-pantie include, but are not limited to, are: deters ruining expensive panties with menstrual and/or vaginal cream stains, saves on excessive laundering, dismisses frustration of unremovable stains from expensive panties, negates re-infecting vaginal areas when vaginal cream treatments are finished, use in high humidity areas for comfort and freshness when needed, when vacationing, reducing the need for excessive laundering, during menstruation and use of vaginal creams and preparations, can be used by hospitals for female patients of varying diagnoses, can be placed in vending machines in female bathrooms in hotels, corporations, and etc.

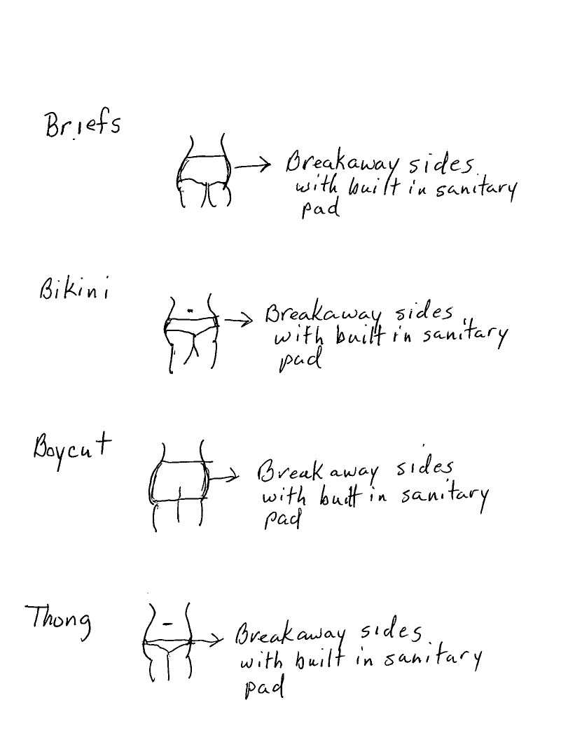

The title of the invention is sani-pantie. I hereby claim priority to the earlier filed provisional application date. Provisional patent application was filed on Mar. 1, 2008, application number 61/067,659.

This is not a federally sponsored research/development

The application includes application transmittal form, fee transmittal form, application data sheet, specifications, drawings, and abstract of disclosure.

The invention is the sole property of Alberta T. Anderson.

The sani-pantie will be designed to be a product consisting of a stretchable, disposable or non disposable mesh cotton, or other materials panty with or without break away sides, and a built in sanitary pad of absorbent cotton, or other materials, with a leak proof underlining of plastic, vinyl, or other materials, and an overlay of thin cotton mesh, or other materials designed to keep wetness away from the vaginal area. The precise invention for which a patent is solicited is the combination of a sanitary napkin and panty manufactured as a single disposable or non disposable unit. The improvement of this product, compared to products of old, is that it eliminates the use of the consumers own expensive underwear with only a sanitary napkin, thus eliminating the risk of ruining the consumers own expensive underwear. It also eliminates the possibility of becoming re-infected by the consumers own underwear after the completed use of pharmaceutical vaginal creams/preparations with the disposable design. Its use can eliminate laundering while on vacation, camping, in high humidity areas, etc., with use of the disposable design. It will be utilized by individual consumers, hospitals, women's clinics, etc.

The drawings reflect the various styles of panties, including but not limited to, brief (1), bikini (2), boycut (3), and thong (4). The drawing does not reflect the esthetics, various colors, with or without design patterns to include, but not limited to flowers, hearts, butterflies, and faieries. It will be designed to include, but not limited to, regular pads, maxi-pads, and panty liners. The sanitary pad will be made in the customary manner, and sewn into the disposable panty, creating a disposable one unit product. The breakaway sides of the panty will be made in the customary manner of pull-up training panties for toddlers.

I, Alberta Thomasina Anderson claim to be the sole inventor of the sani-pantie. It is also claimed that the sani-pantie is the very first of this all inclusive (disposable or non disposable panty and sanitary napkin combination) type of feminine hygiene product, and that there exists no other feminine hygiene product of this specific design.

I hereby claim priority to the earlier filed provisional application date of Mar. 1, 2008, Provisional application number 61/067,659.

1. I claim ownership to this invention and any variation(s) of the sani-pantie filed on or after this date including, but not limited to, any variation(s) of disposable panty consisting of a stretchable, disposable, mesh cotton, or other materials panty, with side panels that are easily separated for convenient removal, with a built in sanitary pad of absorbent cotton, or other materials, with a leak proof underlining of plastic, vinyl, or other materials, and an overlay of thin cotton mesh, or other materials designed to keep wetness away from the vaginal area: The sanitary pad will be made in the customary manner, and sewn into the disposable panty, creating a disposable one unit product. The breakaway sides of the panty will be made in the customary manner of pull-up training panties for toddlers. This product is designed to replace women's expensive panties during menses, use of vaginal pharmaceutical preparations during the vaginal infection process, to prevent re-infection, and for comfort, freshness, and convenience. This includes, but is not limited to stress incontinence, and any gynecological or gastrointestinal disease process. I claim ownership to any prototype, model, or finished product developed for marketing, sales, or personal use, of said invention in the United States and abroad. I, Alberta Thomasina Anderson claim to be the sole inventor of the Sani-Pantie, claiming all subject matter presented herewith in this non provisional patent application, as well as the provisional application, and any future variation(s) of said invention by others.
